SEQUENCE LISTING (4 sequences)

SEQ ID NO: 1 - RNA, 20 nt, Homo sapiens
  source 1..20: mol_type = other RNA
ctacaaagggaagccctttc

SEQ ID NO: 2 - RNA, 23 nt, Homo sapiens
  source 1..23: mol_type = other RNA
tctacaaagggaagccctttctg

SEQ ID NO: 3 - RNA, 22 nt, Homo sapiens
  source 1..22: mol_type = other RNA
aaagtgcttctctttggtgggt

SEQ ID NO: 4 - RNA, 87 nt, Homo sapiens
  source 1..87: mol_type = other RNA
tctcaagctgtgagtctacaaagggaagccctttctgttgtctaaaagaaaagaaagtgcttctctttggtgggttacggtttgaga

(19) United States
(12) Patent Application Publication
(10) Pub. No.: US 2025/0345461 A1
(43) Pub. Date: Nov. 13, 2025

(54) METHOD FOR REPAIRING HAIR CYCLE-RELATED GENES AND METHOD FOR TREATING HAIR CYCLE-RELATED DISEASES USING MIR-520D-5P

(71) Applicant: LIVIUS PTE. LTD., Singapore (SG)

(72) Inventor: Norimasa MIURA, Singapore (SG)

(21) Appl. No.: 18/721,584

(22) PCT Filed: Dec. 16, 2022
(86) PCT No.: PCT/SG2022/050912

§ 371 Date: Jun. 18, 2024

(30) Foreign Application Priority Data
Dec. 29, 2021 (SG) 10202114489Q

(51) Int. Cl. A61K 48/00; A61K 45/06; C07K 14/47; C07K 14/51; C12N 5/00; C12N 5/071; C12N 15/113

(52) U.S. Cl. CPC A61K 48/005; A61K 45/06; C07K 14/4705; C07K 14/51; C12N 5/0018; C12N 5/0627; C12N 15/113; C12N 2310/141; C12N 2501/999

(57) ABSTRACT

It is an object of the present invention to provide a novel means for treating a hair cycle-related disease or symptom (hair cycle-related disease etc. Provided is a miR-520d-p polynucleotide or a polynucleotide encoding thereof or a compound for upregulating miR-520d-5p for use in reverting a mutation in a hair cycle-related gene to wild type in a cell, wherein the polynucleotide comprises: (a1) a nucleotide sequence set forth in SEQ ID NO:1: or (a2) a nucleotide sequence having 1 to 3 nucleotide deletions, substitutions, insertions or additions in the nucleotide sequence set forth in SEQ ID NO:1. SEQ ID NO:1 (single-stranded miRNA sequence) 5′-CUACAAAGGGAAGCCCUUUC-3

### REFERENCE TO RELATED APPLICATIONS

This application claims the priority to Singapore patent application No. 10202114489Q, filed 29 Dec. 2021, the contents of which are incorporated herein by reference.

### TECHNICAL FIELD

The present invention relates to miR-520d-5p polynucleotides and compounds for upregulating miR-520d-5p, especially their novel uses. The present invention also relates to methods and compositions for treating diseases or symptoms (e.g., alopecia) related to the hair cycle.

### BACKGROUND ART

In WO 2012/008302 A, the present inventor disclosed that the use of miR-520d-5p can induce a somatic cell or a malignant tumor cell to a pluripotent stem cell (i.e., an inducing method or an inducing agent), and that the use can also treat malignant tumors (i.e., a therapeutic method or a therapeutic agent).

In WO 2017/073689 A, the present inventor disclosed that the use of miR-520d-5p can ameliorate DNA damage caused by ultraviolet rays (i.e., a composition for ameliorating DNA damage or a composition for ameliorating UV light-induced damage), and that the use can also suppress a decrease in collagen production by UV light (i.e., a composition for promoting collagen production or a composition for suppressing a decrease in collagen production).

In WO2018/203553 A, the present inventor disclosed that the use of miR-520d-5p can improve a quality of a differentiated cell from a stem cell such as an iPS cell (i.e., a method for improving a quality of a differentiated cell).

In WO2017/073692 A, the present inventor disclosed that the use of a HAT1 and/or KAT8 inhibitor can treat malignant tumors, induce stem cells, and ameliorate DNA damage (i.e., a therapeutic agents for malignant tumors, an agent for inducing stem cell, an agent for ameliorating DNA damage) and the like. Examples of HAT1 and/or KAT8 inhibitors include chlorpropamide, vancomycin hydrochloride, betaxolol hydrochloride, cholesterol sulfate, bisoprolol fumarate, pinaverium bromide, oxprenolol hydrochloride, methylbenzethonium chloride, demepotassium bromide, seriprolol hydrochloride, amicacin hydrate, alprenolol hydrochloride and the like.

Documents: Ohyama, M., et al. J. Cell Sci. 125, 4114-4125 (2012); Ceruti, J. M., et al., Mol. Cell Endocrinol. 465, 122-133 (2018); and Lolli, F., et al. Endocrine. 57, 9-17 (2017), each. describe genes expressed in (and related to growth or function of) dermal papilla cells and signaling pathway and the like, and documents Nowak, J. A., et al. Cell Stem Cell. 3, 33-43 (2008); Liu, Y., et al. J. Invest. Dermatol. 121, 963-968 (2003); Rhee, H., et al. Science. 312, 1946-1949 (2006); Horsley, V., et al. Cell. 132, 299-310 (2008); and Xu, Z., et al. Elife. 4, e10567 (2015) describe genes expressed in (and serves as cell markers of) bulge stem cells.

Diseases and symptoms related to hair cycle such as alopecia remain a problem.

### SUMMARY OF INVENTION

Various means (treatment methods, treatment compositions, etc.) for treating diseases or symptoms (e.g., alopecia) related to the hair cycle have been proposed so far, but novel means are still needed to meet the demands of patients who are unable to obtain sufficient advantages by the known means.

It is an object of the present invention to provide a novel means for treating a hair cycle-related disease or symptom (hair cycle-related disease etc.).

The present inventor found that the use of miR-520d-5p can allow a mutation in a gene related to the hair cycle (a hair cycle-related gene) in a cell related to the hair cycle (a hair cycle-related cell) return to the wild type. The present inventor also found that some compounds have the effect of enhancing (upregulating) the expression of miR-520d-5p, and that the use of such compounds for upregulating miR-520d-5p can also restore a mutation in a hair cycle-related gene to the wild type. Furthermore, it was also found that miR-520d-5p is presumed to suppress or regulate the expression of some hair cycle-related genes or other genes in hair cycle-related cells. Accordingly, the present inventor found that a miR-520d-5p polynucleotide or a polynucleotide encoding thereof or a compound for upregulating miR-520d-5p can be used as an active gradient to treat hair cycle-related diseases and the like, and the present inventor has completed the present invention.

That is, the present invention provides at least the following items.

### [Item 1]

In one aspect a miR-520d-5p polynucleotide or a polynucleotide encoding thereof or a compound for upregulating miR-520d-5p for use in reverting a mutation in a hair cycle-related gene to wild type in a cell is provided,
wherein the polynucleotide comprises:
   (a1) a nucleotide sequence set forth in SEQ ID NO:1; or
   (a2) a nucleotide sequence having 1 to 3 nucleotide deletions, substitutions, insertions or additions in the nucleotide sequence set forth in SEQ ID NO:1.

(single-stranded miRNA sequence)
SEQ ID NO: 1
5′-CUACAAAGGGAAGCCCUUUC-3′

### [Item 2]

The miR-520d-5p polynucleotide or the polynucleotide encoding thereof or the compound for upregulating miR-520d-5p according to Item 1, wherein the cell is a dermal papilla cell, a skin-derived precursor cell or a bulge stem cell, or a stem cell that can be differentiated into said cells.

### [Item 3]

The miR-520d-5p polynucleotide or the polynucleotide encoding thereof or the compound for upregulating miR-520d-5p according to Item 1 or 2, wherein the hair life cycle-related gene is at least one gene selected from the group consisting of the following 142 genes:
ACT1, ALPL, ALX4, AQP3, AR, ASCL1,
BCL2, BDNF, BMI1, BMP2, BMP4, BMP6,
CCHCR1, CD271, CDKN1C, CDKN2A, Chr.20 1122 PAX1, CPE, CRHR2, CTGF, CTNNB1, CYP1B1,
DDR2, DKK2, DKK4, DLL1,
EDN3, EFNA, EGF, EGR1, EGR3,
FBN2, FGF5, FGF7 (KGF), FGF18, FN1, FOXE1, FOXN1, FOXO3, FZD1, FZD4, FZD8,
GADD45G, GATA3, GLI2, GLI3, GRID1, GUCY1A3,
HES1, HES5, HEY1, HEY2, HEYL, HGF, HR (HAIRLESS), HSD3B1,
IGF1, IRF4, ITGB1,
JAG1, JAG2,
KAT2A, KAT2B, KCNJ8 (KIR6), KDR, KRT5, KRT14, KRT15, KRT19,
LAMC3, LRIG1, LATS2, LEF1, LFNG, LGR5, LHX2, LMO1, LPL, LRP4, LRP5,
MAPK1,
NCAM1, NCOA3, NDP, NELL2, NFATC1, NFKB, NOG, NOS3, NOTCH2, NOTCH3, NOTCH4, NTRK,
PDGFA, PTCH1,
RBPJ, RGS2, RHOB, RUNX1,
SDKN2A, SERPINE2, SHH, SIRT7, SLC25A37 (MSCP), SMAD4, SMAD6, SMO, SMOC2, SMTN, SOSTDC1, SOX2, SOX9, SPON1, SPRY2, SP5, SRD5A2, STAT3, STS, SUR2 (ABCC9),
TCF7, TFAP2A, TFAP2C, TGFB1, TGFB2, TGFB111, THBS1 (TSP1), TIMP2, TRPS1, TWIST1, TWIST2, TYRO3,
VAV3, VCAN, VDR, VEGFA, VIM, VSIG8, WNT4, WNT5A, WNT7B, WNT9B and WNT10B.

### [Item 4]

The miR-520d-5p polynucleotide or the polynucleotide encoding thereof or the compound for upregulating miR-520d-5p according to any ono of Items 1 to 3, which is used for reverting the mutation in the hair cycle-related gene in the cell to the wild type as well as suppressing or regulating the expression of at least one gene selected from the group consisting of the following 18 genes:
AR,
DIO2,
EDN3,
FGF7 (KGF),
HEY1, HEY2,
LEF1,
NTRK (SLITRK2, 4, 6),
PTHLH,
RNF144A, RORA,
SMOC2, SOSTDC1, SOX9,
TRPS1, TWIST1,
VAV3 and VCAN.

### [Item 5]

The miR-520d-5p polynucleotide or the polynucleotide encoding thereof or the compound for upregulating miR-520d-5p according to any one of Items 1 to 4,
wherein the cell is a dermal papilla cell or a stem cell that can be differentiated into said cell, and
wherein the hair cycle-related gene is at least one gene selected from the group consisting of the following 26 genes:
ALX4, AR,
BMP2, BMP4,
CPE,
EDN3,
FGF7 (KGF),
GUCY1A3,
HEY1, HEY2,
LAMC3, LEF1, LPL, LRP4,
NDP, NOG, NTRK,
SOSTDC1, SPON1,
TFAP2A, TFAP2C, TRPS1, TWIST1,
VAV3, VCAN and WNT5A.

### [Item 6]

The miR-520d-5p polynucleotide or the polynucleotide encoding thereof or the compound for upregulating miR-520d-5p according to any one of Items 1 to 5, which is used for reverting the mutation in the hair cycle-related gene in the cell to the wild type as well as suppressing or regulating the expression of at least one gene selected from the group consisting of VAV3, NTRK (SLITRK2, 4, 6), HEY2, RORA, VCAN, HEY1, AR, TRPS1, TWIST1, LEF1, EDN3, SOSTDC1, FGF7 (KGF), DIO2 and RNF144A.

### [Item 7]

The miR-520d-5p polynucleotide or the polynucleotide encoding thereof or the compound for upregulating miR-520d-5p according to any one of Items 1 to 6,
wherein the cell is a bulge stem cell or a stem cell that can be differentiated into said cell, and
wherein the hair cycle-related gene is at least one gene selected from the group consisting of the following 18 genes:
ASCL1,
BMP2,
DKK4,
GADD45G,
KRT15,
LHX2, LMO1, LRP5,
NFATC1,
SHH, SMOC2, SOSTDC1, SOX2, SOX9, SP5, SRD5A2, STAT3, STS, SUR2 (ABCC9),
TCF7, TFAP2A, TFAP2C, TGFB1, TGFB2, TGFB111, THBS1 (TSP1), TIMP2, TRPS1, TWIST1, TWIST2, TYRO3,
VAV3, VCAN, VDR, VEGFA, VIM, VSIG8, WNT4, WNT5A, WNT7B, WNT9B and WNT10B.

### [Item 8]

The miR-520d-5p polynucleotide or the polynucleotide encoding thereof or the compound for upregulating miR-520d-5p according to any one of Items 1 to 7, which is used for reverting the mutation in the hair cycle-related gene in the cell to the wild type as well as suppressing or regulating the expression of at least one gene selected from the group consisting of PTHLH, SOX9 and SMOC2.

### [Item 9]

The miR-520d-5p polynucleotide or the polynucleotide encoding thereof or the compound for upregulating miR-520d-5p according to any one of Items 1 to 8,
wherein the cells are a dermal papilla cell or a stem cell that can be differentiated into said cell and a bulge stem cell or a stem cell that can be differentiated into said cell, and
wherein the hair cycle-related gene is at least one gene selected from the group consisting of the following 2 genes: BMP2 and SOSTDC1.

### [Item 10]

The miR-520d-5p polynucleotide or the polynucleotide encoding thereof or the compound for upregulating miR-520d-5p according to any one of Items 1 to 9,
wherein the miR-520d-5p polynucleotide is
   a double-stranded polynucleotide constituted by
      a polynucleotide having
      (b1) a nucleotide sequence set forth in SEQ ID NO:2; or
      (b2) a nucleotide sequence having 1 to 3 nucleotide deletions, substitutions, insertions or additions in the nucleotide sequence set forth in SEQ ID NO:2,
      and a polynucleotide having
      (c1) a nucleotide sequence set forth in SEQ ID NO:3; or
      (c2) a nucleotide sequence having 1 to 3 nucleotide deletions, substitutions, insertions or additions in the nucleotide sequence set forth in SEQ ID NO:3, or
   a polynucleotide having
      (d1) a nucleotide sequence set forth in SEQ ID NO:4; or
      (d2) a nucleotide sequence having 1 to 6 nucleotide deletions, substitutions, insertions or additions in the nucleotide sequence set forth in SEQ ID NO:4.

(guide chain sequence)
SEQ ID NO: 2
5′-UCUACAAAGGGAAGCCCUUUCUG-3′
(passenger chain sequence)
SEQ ID NO: 3
5′-AAAGUGCUUCUCUUUGGUGGGU-3′
(Pre-miRNA sequence)
SEQ ID NO: 4
5′-UCUCAAGCUGUGAGUCUACAAAGGGAAGCCCUUUCUGUUGUCUA
AAAGAAAAGAAAGUGCUUCUCUUUGGUGGGUUACGGUUUGAGA-3′

### [Item 11]

The miR-520d-5p polynucleotide or the polynucleotide encoding thereof or the compound for upregulating miR-520d-5p according to any one of Items 1 to 10,
wherein the compound for upregulating miR-520d-5p is selected from the group consisting of the following 13 compounds:
Vancomycin, Metoprolol, Benzethonium, Chlorpropamide, Betaxolol, Colistin, Oxprenolol, Ethacrynic acid, Bisoprolol, Candesartan, Alexidine, Bumetanide and Glycopyrrolate.

### [Item 12]

In another aspect a method for producing a cell is provided, comprising:
1) a step of culturing a cell using the miR-520d-5p polynucleotide or the polynucleotide encoding thereof or the compound for upregulating miR-520d-5p according to any one of Items 1 to 11, and
2) a step of identifying whether a mutation in a hair cycle-related gene is reverted to wild type in the cell.

### [Item 13]

In another aspect a composition for use in treating a hair cycle-related disease or symptom is provided, comprising
(I) the miR-520d-5p polynucleotide or the polynucleotide encoding thereof or the compound for upregulating miR-520d-5p according to any one of Items 1 to 11, or
(II) a cell produced by the method according to Item 12.

### [Item 14]

The composition according to Item 13, for use in therapeutically treating alopecia.

### [Item 15]

The composition according to Item 13 or 14, wherein each of the compounds for upregulating miR-520-5p is administered so as to give the following maximum effective blood concentration or applied externally at the following concentration:
Vancomycin: administration, not more than 30 μg/ml;
Metoprolol: administration, not more than 41.8 ng/ml;
Benzethonium: external application, not more than 0.2%;
Chlorpropamide: administration, not more than 30 μg/ml;
Betaxolol: administration, not more than 41.8 ng/ml;
Colistin: administration, not more than 4.4 μg/ml;
Oxprenolol: administration, not more than 30.18 μg/ml;
Ethacrynic acid: administration, not more than 100 μg/ml;
Bisoprolol: administration, not more than 100 ng/ml;
Candesartan: administration, not more than 75 ng/ml;
Alexidine:
Bumetanide: administration, not more than 0.4 μg/ml;
Glycopyrrolate: administration, not more than 800 μg/ml.

It should be noted that those skilled in the art can change the categories of the above inventions or other inventions included in the present specification to make them different inventions. For example, the invention of “a miR-520d-5p polynucleotide or a polynucleotide encoding the same, or a compound for upregulating miR-520d-5p for use in reverting a mutation in a hair cycle-related gene to the wild type in a cell” may be changed to an invention of “a method for reverting a mutation in a hair cycle-related gene to the wild type in a cell using miR-520d-5p polynucleotides or polynucleotides encoding the same, or a compound for upregulating miR-520d-5p compound”. In addition, the invention of “a composition for use in treating a hair cycle-related disease or symptom comprising a miR-520d-5p polynucleotide or a polynucleotide encoding the same, or a compound for miR-520d-5p upregulation, or a cell produced by the method of the present invention” may be changed to an invention of “a method for treating a hair cycle-related disease or symptom using (administering, etc.) a miR-520d-5p polynucleotide or a polynucleotide encoding the same, or a compound for upregulating miR-520d-5p, or a cell produced by the method of the present invention”. Those skilled in the art can read the matters relating to the invention before the change described in the present specification to the matters relating to the invention after the change.

### Advantageous Effects of Invention

Hair growth and hair loss are closely associated with aging of hair cycle-related cells. Not only aged hair cycle-related cells existing in the living body such as humans, but also iPS cells produced from somatic cells and mesenchymal stem cells derived from the iPS cells are accompanied by many mutations and epigenetic modifications. By using miR-520d-5p, hair cycle-related genes in those cells can be returned to the wild type and normalize the functions of the hair cycle-related cells. Furthermore, miR-520d-5p not only has the action of repairing mutations as described above, but also has the action to suppress or regulate the expression of mRNA having a complementary base sequence, for example, mRNA of a hair cycle-related gene or the other gene that contributes to hair cycle-related diseases. Therefore, by using the miR-520d-5p polynucleotide or the polynucleotide encoding it or the miR-520d-5p upregulating compound in accordance with the present invention, one of the above actions, preferably a synergetic actions of the both can exhibit favorable effects on the treatment of hair cycle-related diseases (e.g., alopecia).

### BRIEF DESCRIPTION OF DRAWINGS

FIG. 1 shows an outline of 178 hair cycle-related genes expressed during the anagen, catagen, telogen and exogen of the hair cycle.

FIG. 2 shows an outline of genes expressed in dermal papilla cells (DP cells) in Example 1.

FIG. 3 shows an outline of genes expressed in bulge stem cells in Example 2.

FIG. 4 is a graph showing the expression level by the compounds for upregulating miR-520d-5p in Example 2.

### DETAILED DESCRIPTION

In various embodiments, in order to return the mutation in the intracellular hair cycle-related gene to the wild type, the miR-520d-5p polynucleotide or the polynucleotide encoding the same, or the miR-520d-5p upregulating compound (these may be collectively referred to as “miR-520d-5p-related substances” herein).

The use of the miR-520d-5p-related substance means that the expression level of miR-520d-5p in the cells is increased more than usual (when the miR-520d-5p-related substance is not used). The expression level of miR-520d-5p can be increased to a desired degree depending on the amount of the miR-520d-5p-related substance introduced into the cells. By using the miR-520d-5p-related substance, the expression level of miR-520d-5p is, for example, 100% or more, 110% or more, 120% or more, 130% or more, 140% or more, 150% or more, 2 times (200%) or more, 3 times (300%) or more, 4 times (400%) or more, 5 times (500%) or more, 10 times (1000%) or more, 20 times (2000%) As mentioned above, it can be 30 times (3000%) or more, 40 times (4000%) or more, or 50 times (5000%) or more, compared with the usual level.

As used herein, the term “hair cycle-related gene” refers to a gene related to the hair cycle and expressed in a cell related to the hair cycle (hair cycle-related cell). The hair cycle includes anagen (the period when hair matrix cells are actively dividing and hair grows fast, usually 2 to 6 years), catagen (the period when hair growth slows down as the function of dermal papilla cells which support the division of hair matrix cells is reduced and the dermal papilla cells move away from the hair matrix cells, usually 2-3 weeks), telogen (the period when dermal papilla cells shrink and hair does not grow, usually 3-4 months), and exogen (the period when hair is missing, usually 2 to 5 months). The hair cycle-related gene is not particularly limited as long as the effect of returning the mutation to the wild type by the miR-520d-5p-related substances is exhibited.

In various embodiments hair cycle-related cells include, for example, dermal papilla cells (DP cells), skin-derived precursor cells (SKP cells) and bulge stem cells, and stem cells that can be differentiated into these cells, such as embryonic stem cells (ES cells) and induced pluripotent stem cells (iPS cells). Dermal papilla cells are classified as mesenchymal stem cells and interact with skin-derived progenitor cells. Bulge stem cells are derived from the peripheral ring of the placode basement membrane and essential for hair growth. Such stem cells also maintain the potential to differentiate into other cell lines important for promoting hair growth, such as blood cells, fibroblasts, adipocytes, and neurons. The hair cycle-related cell may be any one type of cell or may be two or more types of cells.

The target cells (hair cycle-related cells) for returning the mutation in the hair cycle-related gene to the wild type may be those existing in the living body (in vivo) or those existing outside the living body (ex vivo or in vitro). For example, when the treatment method (details are described separately) is carried out, the target cells are present in the living body, and when the cell production method (details are described separately) is carried out, the target cells are present outside the living body.

In various embodiments, the hair cycle-related gene for which the mutation is repaired is at least one gene selected from the group consisting of the following 142 genes:
ACT1, ALPP, ALX4, AQP3, AR, ASCL1,
BCL2, BDNF, BMI1, BMP2, BMP4, BMP6,
CCHCR1, CD271, CDKN1C, CDKN2A, Chr.20 1122 PAX1, CPE, CRHR2, CTGF, CTNNB1, CYP1B1,
DDR2, DKK2, DKK4, DLL1,
EDN3, EFNA, EGF, EGR1, EGR3,
FBN2, FGF5, FGF7 (KGF), FGF18, FN1, FOXE1, FOXN1, FOXO3, FZD1, FZD4, FZD8,
GADD45G, GATA3, GLI2, GLI3, GRID1, GUCY1A3,
HES1, HES5, HEY1, HEY2, HEYL, HGF, HR (HAIRLESS), HSD3B1,
IGF1, IRF4, ITGB1,
JAG1, JAG2,
KAT2A, KAT2B, KCNJ8 (KIR6), KDR, KRT5, KRT14, KRT15, KRT19,
LAMC3, LRIG1, LATS2, LEF1, LFNG, LGR5, LHX2, LMO1, LPL, LRP4, LRP5,
MAPK1,
NCAM1, NCOA3, NDP, NELL2, NFATC1, NFKB, NOG, NOS3, NOTCH2, NOTCH2, NOTCH4, NTRK,
PDGFA, PTCH1,
RBPJ, RGS2, RHOB, RUNX1,
SDKN2A, SERPINE2, SHH, SIRT7, SLC25A37 (MSCP), SMAD4, SMAD6, SMO, SMOC2, SMTN, SOSTDC1, SOX2, SOX9, SPON1, SPRY2, SP5, SRD5A2, STAT3, STS, SUR2 (ABCC9),
TCF7, TFAP2A, TFAP2C, TGFB1, TGFB2, TGFB111, THBS1 (TSP1), TIMP2, TRPS1, TWIST1, TWIST2, TYRO3,
VAV3, VCAN, VDR, VEGFA, VIM, VSIG8, WNT4, WNT5A, WNT7B, WNT9B and WNT10B.

As the hair cycle-related genes, 178 genes can be mentioned as shown in Table 2 of Example 1 described later. In another embodiment, at least one gene selected from genes include in the above 178 genes but not included in the above 142 genes, that is, the following 36 genes (the genes with gray background color in Table 2) may be targeted for repairing a mutation in some cases.

ANGPT, APOE, ASCL4,
BAX,
CD200, CD34, CDH1, CDH3, CLCA2, CSTA,
DIO2, DKK1,
EDA2R, EDAR, ENG (CD105),
FGF2,
GLI1, GPRC5B,
IFITM3, IL6,
LAMA5
MKI67 (Ki67)
NANOGNB (HOXC14), NCOA4
PRSS53, PTGDS, PTHLH,
RNF144A,
SHISA2, SPECC2, SPP1, SPRY1, SRD5A1, STAR, STAT2 and
WIF1.

In various embodiments, the miR-520d-5p-related substance can repair mutations in hair cycle-related genes and suppress or regulate the expression of a gene (mRNA) containing a base sequence having a certain complementarity with miR-520d-5p among those expressed in cells, preferably hair cycle-related cells. Genes to be suppressed or regulated by miR-520d-5p can be estimated by using known miRNA target search databases or websites (e.g., miRBase, EumiR, miRanda, PicTar, TargetScanHuman 7.0). For example, genes with a target score of 50 or higher, 60 or higher, 70 or higher, 80 or higher, or 90 or higher may be targeted. The gene whose expression is to be suppressed or regulated may be a hair cycle-related gene or another gene, and for example, at least one gene selected from the group consisting of the following 18 genes:
AR,
DIO2,
EDN3,
FGF7 (KGF),
HEY1, HEY2,
LEF1,
NTRK (SLITRK2, 4, 6),
PTHLH,
RNF144A, RORA,
SMOC2, SOSTDC1, SOX9,
TRPS1, TWIST1,
VAV3 and VCAN.

“Suppressing” gene expression by miR-520d-5p (related substance) means that the expression level of the target gene in the cell is reduced as compared with the case where the miR-520d-5p related substance is not used. The expression level of the target gene can be suppressed to a desired degree depending on the amount of the miR-520d-5p-related substance introduced into the cell, etc., and for example, as compared with the case where the miR-520d-5p related substance is not used, it can be less than 100%, 90% or less, 80% or less, 70% or less, 60% or less, 50% or less, 40% or less, 30% or less, 20% or less, 10% or less, 5% or less, 1% or less, 0.1% or less, and 0.01% or less. On the other hand, “regulating” gene expression by miR-520d-5p (related substance) means keeping the expression level of the target gene in cells within a certain range from the expression level in normal cells. The expression level of the target gene can be regulated to a desired degree depending on the amount of the miR-520d-5p-related substance introduced into the cell, etc., and for example, as compared with the expression level in normal cells, ±100% or less, ±50% or less, ±20% or less, ±10% or less, or ±5% or less. An embodiment in which the expression level in a cell is kept within a certain range as a result of applying miR-520d-5p (related substance) to the cell in which the target gene is overexpressed and “suppressing” (reducing) the expression level of the gene also corresponds to “suppressing or regulating” the expression of a gene by miR-520d-5p (related substance).

In various embodiments, the hair cycle-related gene for which the mutation is repaired is at least one gene expressed in a dermal papilla cell or a stem cell capable of differentiating into the cell, and is selected from the group consisting of the following 26 genes:
ALX4, AR,
BMP2, BMP4,
CPE,
EDN3,
FGF7 (KGF),
GUCY1A3,
HEY1, HEY2,
LAMC3, LEF1, LPL, LRP4,
NDP, NOG, NTRK,
SOSTDC1, SPON1,
TFAP2A, TFAP2C, TRPS1, TWIST1,
VAV3, VCAN and WNT5A.

In addition to the above 26 types of genes, there are 7 types of hair cycle-related genes (APOE, CLCA2, CSTA, DIO2, GPRC5B, RNF144A, WIF1) expressed in dermal papilla cells. In another embodiment, for example, when cells cultured under different conditions from the Examples disclosed herein and having different mutations are targeted, at least one gene of these 7 types of genes may be targeted for repairing the mutations in some cases.

Further, in the above embodiment in which dermal papilla cells are target cells, in addition to the above 26 types of genes (or another 7 types of genes), at least one gene of 6 types of genes (ALPL, CCND2, CTSH, FRZB, PTGS2, RORA) expressed in dermal papilla cells but not corresponding to the hair cycle-related genes may be targeted for repairing the mutations.

Furthermore, in the above embodiment in which dermal papilla cells are the target cells, the use of miR-520d-5p can repair mutations in the specific genes, and at the same time, the use can also suppress or regulate the expression of a specific gene (mRNA) among the genes expressed in dermal papilla cells. The genes expressed in dermal papilla cells that can be the target of such expression suppression or regulation include, for example, at least one gene selected from the group consisting of VAV3, NTRK (SLITRK2, 4, 6), HEY2, RORA, VCAN, HEY1, AR, TRPS1, TWIST1, LEF1, EDN3, SOSTDC1, FGF7 (KGF), DIO2 and RNF144A. In particular, VAV3, NTRK, HEY2, VCAN, HEY1, AR, TRPS1, TWIST1, LEF1, EDN3, SOSTDC1 and FGF7 (KGF) can be subjected to m both mutation repairing and suppression or regulation of expression by miR-520d-5p. Among them, since AR is a gene overexpressed in androgenetic alopecia (AGA) and female androgenetic alopecia (FAGA), AR is important as the hair cycle-related gene expressed in hair papilla cells.

In various embodiments, the hair cycle-related gene for which the mutation is repaired is at least one gene expressed in a bulge stem cell or a stem cell capable of differentiating into the cell, and is selected from the group consisting of the following 18 genes:
ASCL1,
BMP2,
DKK4,
GADD45G,
KRT15,
LHX2, LMO1, LRP5,
NFATC1,
SHH, SMOC2, SOSTDC1, SOX2, SOX9, SP5, SRD5A2, STAT3, STS, SUR2 (ABCC9),
TCF7, TFAP2A, TFAP2C, TGFB1, TGFB2, TGFB111, THBS1 (TSP1), TIMP2, TRPS1, TWIST1, TWIST2, TYRO3,
VAV3, VCAN, VDR, VEGFA, VIM, VSIG8, WNT4, WNT5A, WNT7B, WNT9B and WNT10B.

In addition to the above 18 types of genes, there are 5 types of hair cycle-related genes (CDH3, EDAR, IFITM3, PTHLH, WIFI) expressed in bulge stem cells. In another embodiment, for example, when cells cultured under different conditions from the Examples disclosed herein and having different mutations are targeted, at least one gene of these 5 types of genes may be sometimes targeted for repairing the mutations in some cases.

Furthermore, in the above embodiment in which bulge stem cells are the target cells, the use of miR-520d-5p can repair mutations in specific genes, and at the same time, the use can also suppress or regulate the expression of a specific gene (mRNA) among the genes expressed in dermal papilla cells. The genes expressed in bulge stem cells that can be the target of such expression suppression or regulation include, for example, at least one gene selected from the group consisting of PTHLH, SOX9 and SMOC2. In particular, SOX9 can be subjected to both mutation repairing and suppression or regulation of expression by miR-520d-5p, thus SOX9 is important as the hair cycle-related gene expressed in bulge stem cells.

In various embodiments, the hair cycle-related gene for which the mutation is repaired is a gene expressed in both a dermal papilla cell or a stem cell capable of differentiating into the cell and a bulge stem cell or a stem cell capable of differentiating into the cell, and is at least one gene selected from the group consisting of the following two genes:
BMP2 and SOSTDC1.

As used herein, the term “converting a mutation in a hair cycle-related gene to the wild type” means that the base sequence of a hair cycle-related gene different from the wild type (that is, mutated) in the cell is repaired to be the same base sequence as the wild type. The number of mutated bases may be, for example, in a range defined by an arbitrary lower limit value and/or an arbitrary upper limit value selected from the group consists of 1 base, 5 bases, 10 bases, 20 bases, 30 bases, 40 bases, 50 bases, 60 bases, 70 bases, 80 bases, 90 bases, 95 bases and 100 bases (the upper limit value and/or the lower limit value itself may or may not be included), e.g., 1 base or more and 100 bases or less. The bases to be converted may be all or a part of the mutated bases, for example, the conversion rate (the ratio of the number of converted bases to the mutated bases) may be, for example, in a range defined by an arbitrary lower limit value and/or an arbitrary upper limit value selected from the group consisting of 1%, 5%, 10%, 20%, 30%, 40%, 50%, 60%, 70%, 80%, 90%, 95% and 100% (the upper limit value and the lower limit value itself may or may not be included), e.g., 1% or more and 100% or less. Such action and effect may be observed in one cell (e.g., a hair cycle-related cell), or may be observed in a certain proportion (preferably 50% or more) of cells in a cell population containing a certain number of cells.

### MiR-520d-5p Polynucleotide/Polynucleotide Encoding Thereof

As used herein, the term “miR-520d-5p polynucleotide” may refer to a polynucleotide having (a1) a base sequence of SEQ ID NO: 1 or (a2) a base sequence having deletion, substitution, insertion, or addition of several bases in the base sequence of SEQ ID NO: 1. The “several” in the base sequence (a2) is preferably 1 to 3, more preferably 1 to 2, and even more preferably 1.

(single-stranded miRNA sequence)
SEQ ID NO: 1
5′-CUACAAAGGGAAGCCCUUUC-3′

As used herein, the term “polynucleotide encoding the miR-520d-5p polynucleotide” refers to a polynucleotide capable of expressing a polynucleotide having a base sequence (a1) or (a2), that is, capable of producing a polynucleotide by transcription, typically a vector such as an expression vector (a plasmid vector), a viral vector, and the like. The vector may be double-stranded or single-stranded, and may be DNA or RNA. The vector may be transiently present or persistently present by replication in the nucleus or cytoplasm, or may be integrated into genomic DNA and permanently present. Examples of the plasmid vector include those prepared based on Escherichia coli-derived plasmid, Bacillus subtilis-derived plasmid, yeast-derived plasmid, and bacteriophage such as k phage. Examples of the virus vector include a lentivirus vector, an adenovirus vector, an adeno-associated virus (AAV) vector, and the like, which can be produced by a known method.

In various embodiments, the “polynucleotide” may be composed of the nucleotides constituting the natural RNA molecule or DNA molecule, that is, adenine (A), guanine (G), cytosine (C), uracil (U) and thymine (T), or may be composed of nucleotides and nucleotide analogs, nucleotide derivatives, and other nucleotide equivalents. For example, for the purpose of improving the nuclease resistance of polynucleotides, stabilizing, increasing the affinity with complementary-stranded nucleic acids, avoiding off-target effects, increasing transport efficiency to target cells or cell permeability, or visualizing, the polynucleotide may contain a nucleotide analog or a nucleotide derivative.

The nucleotide analog may be a natural molecule or a non-natural molecule, and examples thereof include a nucleoside-modified nucleotide analog, a sugar moiety-modified nucleotide analog, and a phosphate diester bond-modified nucleotide analog.

Specific examples of the nucleoside-modified nucleotide analog include a nucleotide derivative in which the nucleoside moiety (purine ring, pyrimidine ring) is replaced with a group derived from an aromatic compound such as a benzene derivative or a pyridine derivative.

Specific examples of sugar-modified nucleotide analogs include nucleotide analogs substituted with 2′-O-methylribose, 2′-O-propylribose, 2′-methoxyethoxyribose, 2′-O-methoxyethylribose, 2′-O-[2-(guanidium) ethyl] ribose or 2′-fluororibose; nucleotide analogs with the sugar moiety substituted a morpholino ring; and bridged-structured nucleotide analogs with a bridged structure introduced into the sugar moiety (Bridged Nucleic Acid: BNA), for example, Locked Nucleic Acid (LNA) having a structure in which an oxygen atom at the 2′position and a carbon atom at the 4′position are crosslinked with a methylene group, and Ethylene-bridged Nucleic Acid (ENA) having a structure in which an oxygen atom at the 2′position and a carbon atom at the 4′position are crosslinked with an ethylene group.

Specific examples of the phosphate diester bond modified nucleotide analog include nucleotide analogs in which the phosphate diester bond is substituted with a phosphorothioate bond or an N3′-P5′ phosphoamidate bond.

Specific examples of the nucleotide derivative include molecules obtained by adding another chemical substance to a nucleotide, for example, DNA-added derivatives, 5′-polyamine-added derivatives, cholesterol-added derivatives, steroid-added derivatives, bile acid-added derivatives, vitamin-added derivatives, Cy5-added derivatives, Cy3 addition derivatives, 6-FAM addition derivatives, and biotin addition derivatives and the like.

In various embodiments, the miR-520d-5p polynucleotide may be a polynucleotide consisting of nucleotides corresponding to the base sequence (a1) or (a2), or a polynucleotide containing a nucleotide corresponding to the base sequence (a1) or (a2) and a nucleotide having another base sequence. For example, when the miR-520d-5p polynucleotide takes the form of a mature miRNA or a pre-miRNA described below, the miR-520-5p may contain the portion consisting of nucleotides corresponding to the base sequence (a1) or (a2) and the portion consisting of nucleotides corresponding to another sequence contained in the mature miRNA or the pre-miRNA.

A polynucleotide consisting of nucleotides corresponding to the base sequence (a1) or (a2) is incorporated into the RISC complex as a single-stranded miRNA and degrades an mRNA having a base sequence 100% complementary to the single-stranded miRNA. Alternatively, the polynucleotide inhibits the translation of mRNA having a base sequence that is incompletely complementary to the single-stranded miRNA. Based on the online algorithm analysis (miRBase—http://www.mirbase.org/; RNAcentral—https://rnacentral.org/; TargetScanHuman—http://www.targetscan.org/vert_72/), the polynucleotide having a sequence of SEQ ID NO: 1 (natural miR-520d-5p) is expected to bind to more than 9000 mRNAs.

In various embodiments, the “miR-520d-5p polynucleotide” may be a double-stranded polynucleotide (mature miRNA) composed of a polynucleotide having (b1) a base sequence of SEQ ID NO: 2 (guide chain sequence) or (b2) a base sequence having deletion, substitution, insertion, or addition of several bases in the base sequence of SEQ ID NO: 2 and a polynucleotide having (c1) a base sequence of SEQ ID NO: 3 (passenger chain sequence) or (c2) a base sequence having deletion, substitution, insertion, or addition of several bases in the base sequence of SEQ ID NO: 3. Each of the “several” in the base sequence (b2) and (c2) is preferably 1 to 3, more preferably 1 to 2, and even more preferably 1.

(guide chain sequence)
SEQ ID NO: 2
5′-UCUACAAAGGGAAGCCCUUUCUG-3′
(passenger chain sequence)
SEQ ID NO: 3
5′-AAAGUGCUUCUCUUUGGUGGGU-3′

In various embodiments, the “miR-520d-5p polynucleotide” is a polynucleotide having (d1) a base sequence of SEQ ID NO: 4 (pre-miRNA sequence) or (d2) a base sequence having deletion, substitution, insertion, or addition of several bases in the base sequence of SEQ ID NO: 4 The “several” in the base sequence (d2) is preferably 1 to 6, more preferably 1 to 4, still more preferably 1 to 2, and particularly preferably 1.

(Pre-miRNA sequence)
SEQ ID NO: 4
5′-UCUCAAGCUGUGAGUCUACAAAGGGAAGCCCUUUCUGUUGUCUA
AAAGAAAAGAAAGUGCUUCUCUUUGGUGGGUUACGGUUUGAGA-3′

The base length of the miR-520d-5p polynucleotide is not particularly limited, and can be adjusted within the range in which the action and effects are exhibited. For example, when the miR-520d-5p polynucleotide consists of nucleotides corresponding to the base sequence (a1), (b1), (c1) or (d1), the polynucleotide may have a base length of 20, 23, 22, or 87 matching the each base sequences, and when the polynucleotide consists of nucleotides corresponding to the base sequence (a2), (b2), (c2) or (d2), the polynucleotide may have a base length of a number derived from deletion, substitution, insertion, or addition of several bases to 20, 23, 22, or 87.

Compound for Upregulating miR-520d-5p

As used herein the term “compound for upregulating miR-520d-5p” refers to a compound having an action of enhancing (upregulating) the expression of miR-520d-5p polynucleotide in a cell. The compound having such an action is not particularly limited, and can be selected from various compounds (preferably, compounds that has already been used as an active ingredient for pharmaceuticals for other indications and is regarded as relatively safe) and used herein. As the compound for upregulating miR-520d-5p, any one type may be used, or two or more types may be used in combination.

Specific examples of the compound for upregulating miR-520d-5p include the following 13 compounds: vancomycin, metoprolol, benzethonium, chlorpropamide, betaxolol, colistin, oxprenolol, etacrynic acid, bisoprolol, candesartan, alexidine, bumetanide and glycopyrrolate. Any one of these 13 compounds may be used, or two or more kinds may be used in combination.

The compound for upregulating miR-520d-5p may form a pharmaceutically acceptable salt. The pharmaceutically acceptable salt may be selected from, for example, metal salts, ammonium salts, salts with organic base, salt with inorganic acid, salts with organic acid, salts with basic or acidic amino acid, halides and the like in consideration of the chemical structure etc. of the compound for upregulating miR-520d-5p. Examples of the metal salt include alkali metal salts (sodium salt, potassium salt, etc.), alkaline earth metal salts (calcium salt, magnesium salt, barium salt, etc.), and aluminum salts. Examples of the salt with the organic base include salts with trimethylamine, triethylamine, pyridine, picoline, 2,6-lutidine, ethanolamine, diethanolamine, triethanolamine, cyclohexylamine, dicyclohexylamine, N, N′-dibenzylethylenediamine. Examples of the salt with the inorganic acid include salts with hydrochloric acid, hydrobromic acid, nitric acid, sulfuric acid, and phosphoric acid. Examples of salts with organic acid include formic acid, acetic acid, trifluoroacetic acid, phthalic acid, fumaric acid, mesylic acid, tosylic acid, oxalic acid, tartaric acid, maleic acid, citric acid, succinic acid, malic acid, methanesulfonic acid, benzenesulfonic acid, or p-toluenesulfonic acid. Examples of the salt with basic amino acid include salts with arginine, lysine or ornithine, and examples of the salt with acidic amino acid include salts with aspartic acid or glutamic acid. Examples of the halide include chloride, bromide, and iodide. Preferable salts of vancomycin include, for example, hydrochloride. Preferable salts of metoprolol include, for example, tartrate. Preferable salts of benzethonium include, for example, chloride. Preferable salts of betaxolol include, for example, hydrochloride. Preferable salts of colistin include, for example, sulfate. Preferable salts of oxprenolol include, for example, hydrochloride. Preferable salts of bisoprolol include, for example, fumarate.

The compound for upregulating miR-520d-5p or the pharmaceutically acceptable salt thereof may be a solvate. The solvates may be variable among the compound for upregulating miR-520d-5p or the pharmaceutically acceptable salt thereof and depending on a solvent in which the compound or the like is dissolved, or an atmosphere in which the compound or the like is placed (for example, the air with humidity to some extent constant), and examples thereof include hydrates (monohydrate, dihydrate, trihydrate, etc.), ethanol solvates, acetic acid solvates, and the like.

### Cell Production Method

In various embodiments the cell production method comprises (1) a step of culturing a cell using a miR-520d-5p polynucleotide or a polynucleotide encoding the same or a compound for upregulating miR-520d-5p (miR-520d-5p-related substance) (hereinafter referred to as “culture step”), and (2) a step of confirming whether a mutation in a hair cycle-related gene in the cell has returned to the wild type (hereinafter referred to as “confirmation step”).

The culture step can be performed by culturing the target cell (hair cycle-related cell) in a medium to which a miR-520d-5p-related substance has been added. The period of the culture step can be adjusted within a range in which the action and effects of restoring the wildtype or partially restoring the wildtype is exhibited, depending on the type of the target cell and the like, and is not particularly limited. The period of the culture step may be, for example, in a range defined by an arbitrary lower limit value and/or an arbitrary upper limit value selected from the group consists of 1 day, 2 days, 4 days. 6 days, 7 days (1 week), 8 days, 10 days, 12 days, 14 days (2 weeks), 16 days, 18 days, 20 days, 21 days (3 weeks), 22 days, 24 days, 26 days, 28 days (4 weeks) and 30 days (the upper limit value and/or the lower limit value itself may or may not be included), e.g., 6 days or 7 days (1 week) or more and 21 days (3 weeks) or 22 days or less. The medium to which the miR-520d-5p-related substance is added may be used in the entire period of the culture step, or a medium to which the miR-520d-5p-related substance is added may be used in a part of the period of the culture step and a medium to which the miR-520d-5p-related substance is not added may be used during the remaining period of the culture step.

The concentration of the miR-520d-5p-related substance in the medium of the culture step can be adjusted depending on the type of the used miR-520d-5p-related substance within the range in which the action and effects of restoring the wildtype or partially restoring the wildtype is exhibited, and is not particularly limited.

The culture medium, that is, the type of basal medium, and the types and concentrations of the components other than the miR-520d-5p-related substance added thereto can be appropriately adjusted according to the type of the target cell and the like, and is not particularly limited.

In the culture step, known means for improving the efficiency of introducing the miR-520d-5p-related substance into the target cell may be used. For example, miR-520d-5p-related substances, particularly a miR-520d-5p polynucleotide or a polynucleotide encoding the same can be introduced into the target cell in the form of a plasmid vector by electroporation, microinjection, lipofection, or other transfection methods (e.g., by adding a lipofection reagent to the medium), or in the form of a virus vector by infection to the cell.

The confirmation step can be performed by determining the base sequence of the hair cycle-related gene in the cell (target base sequence), comparing the target base sequence with the wild-type base sequence, and determining that the target base sequence has returned to the wild-type if the two base sequences match. The base sequence of the hair cycle-related gene can be determined by using known means. For the wild-type base sequence, those registered in the database can be referred to.

The hair cycle-related gene to be confirmed may be one type or two or more types depending on the purpose of the confirmation step and/or the cell production method, but for example, depending on the cell type, the gene may be all or part of the hair cycle related genes in the specific embodiment as described above. In various embodiments, in the confirmation step, the proportion of hair cycle-related genes that have returned to the wild type among the hair cycle-related genes that have been set in advance for the target cells obtained in the culture step is determined, and it is checked if the proportion is reached a certain level.

The number of target cells may be one or two or more depending on the purpose of the confirmation step and/or the cell production method. In various embodiments, in the confirmation step, a part of the cell population obtained in the culture step is used as a sample, and among the target cells contained therein, the proportion of cells in which mutations in the hair cycle-related genes are returned to the wild-type (or the proportion of cells in which the proportion of hair cycle-related genes that have returned to the wild type among the hair cycle-related genes that have been set in advance is reached a certain level) is determined, and it is checked if the proportion is reached a certain level.

The cell production method may include a step other than the culture step and the recovery step, if necessary. Examples of such an optional step include a step of recovering cells whose target base sequence has returned to the wild-type base sequence (hereinafter referred to as “recovery step”).

In the recovery step, cells whose target base sequence has returned to the wild-type base sequence can be recovered by using known means. Alternatively, the entire cell population in which the proportion of cells in which mutations in intracellular hair cycle-related genes have returned to the wild type has reached a certain level (which can be regarded as such by measurement using a sample) may be recovered.

In one aspect, a method for repairing a hair cycle-related gene is provided, comprising culturing a cell using a miR-520d-5p polynucleotide or a polynucleotide encoding the same or a compound for upregulating miR-520d-5p (miR-520d-5p-related substance) to return a mutation in a hair cycle-related gene to the wild type in the cell.

### Treatment Method/Composition

In various embodiments, the method for treating a hair cycle-related disease or symptom comprises administering (I) a miR-520d-5p polynucleotide or a polynucleotide encoding the same, or a compound for upregulating miR-520d-5p (miR-520d-5p-related substance), or (II) cells obtained by the method.

In various embodiments the composition is used for treating a hair cycle-related disease or symptom (that is, carrying out the treatment method described herein above), and contains (I) miR-520d-5p polynucleotide or a polynucleotide encoding the same, or a compound for upregulating miR-520d-5p (miR-520d-5p related substance), or (II) cells obtained by the method described herein above.

By administering the miR-520d-5p-related substance (I), the action and effects of the miR-520d-5p-related substance of restoring the wildtype or partially restoring the wildtype is exerted on the hair cycle-related cells in vivo, and the hair cycle-related disease or symptom can be treated. Hair cycle-related diseases or symptoms can also be treated by administering the cells (II) obtained by the method described herein above, that is, cells that have already exhibited the effect of returning mutations in cycle-related genes to the wild type. In the treatment method and the composition, as the miR-520d-5p-related substance (I), either one of the miR-520d-5p polynucleotide or the polynucleotide encoding the same, and the compound for upregulating miR-520d-5p may be used, or both may be used (sequentially or simultaneously). Further, either the miR-520d-5p-related substance (I) or the cells (II) obtained by the method described herein above may be used, or both may be used (sequentially or simultaneously).

The “hair cycle-related disease” refers to a disease related to anagen, catagen, telogen or exogen contained in the hair cycle, particularly a disease related to the expression (suppression or enhancement) of a hair cycle-related gene in dermal papilla cells, skin-derived progenitor cells or bulge stem cells, or stem cells capable of differentiating into those cells. The hair cycle-related disease which is the target of the treatment of the symptom by the composition is not particularly limited, but alopecia is typically mentioned. Alopecia generally has a shorter anagen (e.g., months to a year) and a relatively longer catagen and telogen phase, resulting in symptoms such as hair loss, softening of the hair (thick and long hair does not regenerate and most of the hair becomes thin and short) and weakening of the hair, etc., and includes, for example, male alopecia, female alopecia, circular alopecia, trichotyromania, telogen hair loss, hair loss due to endocrine abnormalities, hair loss due to nutritional disorders, hair loss due to skin infections, hair loss due to skin tumors, scarring hair loss, hair loss due to drugs or chemical substances, and various hair malformations.

As used herein, the term “treatment” means completely or partially therapeutically treating a hair cycle-related disease, completely or partially curing (alleviating or ameliorating) the symptoms associated with the hair cycle-related disease, slowing the progression of, delaying the development of, prevent of, or reducing the risk of a hair cycle-related disease or its symptoms, or a combination thereof.

The subject of the treatment may be a human or a non-human animal, and the sex and age are not particularly limited. Human subjects include, for example, patients with hair cycle-related diseases or those who wish to prevent hair cycle-related diseases. Non-human animals include, for example, non-human mammals such as mouse, rat, rabbit, cow, monkey, chimpanzee, pig, horse, sheep, goat, dog, cat, guinea pig and hamster, and preferably mammals that can serve as a model for hair cycle-related diseases. The subject of treatment may be a subject in a nonclinical or clinical trial.

The dose of the miR-520d-5p-related substance or the cells obtained by the method described herein above can be adjusted within the range in which the action and effect of restoring the wildtype or partially restoring the wildtype are exhibited, and is not particularly limited.

In various embodiments, the compound for upregulating miR-520d-5p can be administered in an amount such that the maximum effective blood concentration shown in Table 1 is obtained, or can be used as an external preparation having the concentration shown in Table 1. The maximum effective blood concentration at the time of administration or the concentration in the external preparation of the compound for upregulating miR-520d-5p can be the same (sa) or lower (e.g., 100% or less, less than 100%, 90% or less, 80% or less, 70% or less, 60% or less, 50% or less, 40% or less, It can be 30% or less, 20% or less, 10% or less, 5% or less, 1% or less, 0.1% or less, 0.01% or less) than the maximum effective blood concentration at the time of administration or the concentration in the external preparation when the same compound is used as an active ingredient for other known indications.

TABLE 1

| | Maximum effective blood | Maximum effective blood |
|---|---|---|
| | concentration at the time of | concentration at the time of |
| | administration or | administration or concentration in |
| | concentration in an external | an external preparation in known |
| Compound | preparation | indications |
| Vancomycin | Administration: not | MRSA infection |
| (e.g., hydrochloride) | more than 30 μg/mL | Administration: 30 μg/mL |
| Metoprolol | Administration: not | Hypertension |
| (e.g., tartate) | more than 41.8 ng/mL | Administration: 41.8 ng/mL |
| Benzethonium | External application: | Skin and mucous membrane |
| (e.g., chloride) | not more than 0.2% | infections |
| | | External application: |
| | | 0.3% (1.93 μg/mL) |
| Chlorpropamide | Administration: not | Type 2 diabetes |
| | more than 30 μg/mL | Administration: 30 μg/mL |
| Betaxolol | Administration: not | Essential hypertension, |
| (e.g., hydrochloride) | more than 41.8 ng/mL | parenchymal renal hypertension, |
| | | angina, glaucoma, ocular |
| | | hypertension |
| | | Administration: 41.8 ng/mL |
| Colistin | Administration: not | Escherichia coli, Shigella, |
| (e.g., sulfate) | more than 4.4 μg/mL | multidrug-resistant Pseudomonas |
| | | aeruginosa, infectious enteritis |
| | | Administration: 4.4 μg/mL |
| Oxprenolol | Administration: not | Tachyarrhythmia, angina |
| (e.g., hydrochloride) | more than 30.18 μg/mL | Administration: 30.18 μg/μL |
| Ethacrynic acid | Administration: not | Edematous diseases |
| | more than 100 μg/mL | Administration: 100 μM |
| Bisoprolol | Administration: not | Chronic heart failure due to |
| (e.g., fumarate) | more than 100 ng/mL | essential hypertension, angina, |
| | | ischemic heart disease, and |
| | | dilated cardiomyopathy |
| | | Administration: 100 ng/mL |
| Candesartan | Administration: not | Hypertension |
| | more than 75 ng/mL | Administration: 75 ng/mL |
| Alexidine | External application: | Bacterial and fungal infections |
| | not more than 4.5 ppm | External application: 4.5 ppm |
| | | Inhibition of cancer growth: 1 mM |
| Bumetanide | Administration: not | Edema, heart failure |
| | more than 0.4 μg/mL | Administration: 0.4 μg/mL |
| Glycopyrrolate: | Administration: not | Irritable bowel syndrome/pre- |
| | more than 800 pg/mL | anesthesia treatment/ |
| | | bronchiolitis obstruction |
| | | Administration: 800 pg/mL |

The amount (concentration) of the active ingredient contained in the composition, that is, the miR-520d-5p-related substance or the cells obtained by the method described herein above can be adjusted within the range in which the action and effects of restoring the wildtype or partially restoring the wildtype are exhibited, for example, in consideration of the dosage form, administration route, number of times (frequency) of administration and the like, and is not particularly limited. Appropriate usage, dose, etc. of the miR-520d-5p-related substance or the cells obtained by the method described herein above may be set through non-clinical tests (cell test, animal test) and clinical test.

The dosage form and administration route of the composition can be appropriately selected, and for example, an external preparation to be directly and transdermally or transcutaneously administered (applied) to the affected area (scalp, etc.) or an injection locally administered are suitable. By preparing the composition an external preparation for transdermal administration, an injection for local administration, etc., the active ingredient can be effectively acted around the affected area, and the effect by the treatment can be exerted at a relatively low concentration; it is advantageous in terms of cost, and side effects that are a concern when the dosage form for systemic administration containing a high concentration of the active ingredient can be avoided. On the other hand, when side effects are not substantially a problem, the composition may be in a dosage form for systemic administration by intravenous injection or the like, or for oral administration.

Examples of the dosage form of the external skin or mucous membrane preparation include liquids, sprays, ointments, creams, gels, patches and the like. When the external skin preparation is (medicinal) cosmetics, for example, a hair care product such as shampoo, conditioner, hair styling product (hair tonic, hair conditioner, hair treatment, hair lotion, hair cream, hair oil, hair gel, hair spray) can be mentioned.

The injection may be an intravenous injection, an intraarterial injection, an intramuscular injection, an intradermal injection, a subcutaneous injection and the like. When the cells obtained by the method described herein above are administered, the composition may be administered as a cell preparation by infusion or injection.

Examples of the dosage form of the oral preparation include tablets, capsules, granules, powders, fine granules, syrups, pills, liquids and the like, and the release may be controlled such as sustained release.

Ingredients other than the active ingredient contained in the composition may include additives commonly used in the production of pharmaceuticals, quasi-drugs, cosmetics and the like, preferably additives used in the production of external preparations, injections, oral preparations and the like, for example, stabilizers, preservatives, buffers, pH adjusters, excipients, bases, etc. The content of such additives can also be appropriately adjusted by those skilled in the art.

In addition, to the treatment method and composition described herein above, it is possible to apply technical matters (preferable embodiments, etc.) known regarding pharmaceuticals using a nucleic acid such as miRNA, a so-called oligonucleotide therapeutics and the like.

For example, in the composition it is preferable that the miR-520d-5p-related substance is in a state to which a technology related to the drug delivery system (DDS) used for injections, external preparations and the like is applied. One specific example of DDS is nanoparticle formation, that is, inclusion of miR-520d-5p-related substances in nano-sized particles composed of a specific compound to improve delivery efficiency to target cells. The nanoparticles may contain, as the miR-520d-5p-related substance, only the miR-520d-5p polynucleotide or the polynucleotide encoding the same, or only the compound for upregulating miR-520d-5p (e.g., benzethonium), or both the polynucleotide encoding the miR-520d-5p polynucleotide or the polynucleotide encoding the same and the compound for upregulating miR-520d-5p. Alternatively, the nanoparticles preparation may comprise a plurality of types of nanoparticles, for example, both nanoparticles containing a miR-520d-5p polynucleotide or a polynucleotide encoding the same, and nanoparticles containing a compound for upregulating miR-520d-5p A variety of compounds constituting the nanoparticles are known and are not particularly limited, and examples thereof include biocompatible lipids (e.g., triglycerides, diglycerides, monoglycerides, fatty acids, steroids), surfactants (e.g., phospholipids), polymers (e.g., polylactic acid: PLA, polylactic acid glucolic acid copolymer: PLGA, polyethylene glycol: PEG) and the like. Considering the types of miR-520d-5p-related substances, solubility (water-soluble/fat-soluble), other properties, etc., and using appropriate raw materials and manufacturing methods, nanoparticles having an appropriate inclusion ratio and appropriate size (particle size distribution) can be produced.

In the composition, the miR-520d-5p-related substance may be formulated into collagen, that is, may be included in a base (filler) comprising collagen. In other words, the composition may take the form of a collagen preparation containing a miR-520d-5p-related substance and a based (filler) containing collagen. Since the collagen preparation is gradually absorbed in the living body over a certain period of time after being injected intradermally or subcutaneously, the miR-520d-5p-related substance included in the collagen can be gradually released. The collagen preparation may contain, as the miR-520d-5p-related substance (I), only the miR-520d-5p polynucleotide or the polynucleotide encoding the same, or only the compound for upregulating miR-520d-5p, or both the polynucleotide encoding the miR-520d-5p polynucleotide or the polynucleotide encoding the same and the compound for upregulating miR-520d-5p. In addition, the collagen preparation may contain the cells (II) obtained by the method described herein above. The base of the collagen preparation contains collagen such as type I collagen and type III collagen derived from humans or non-human animals (preferably derived from humans when administered to humans), and if necessary, others components, for example, hyaluronic acid, gelatin (denatured collagen) and the like.

The composition can be produced as various composition in accordance with to the legal system, guidelines, etc. of each country and classification such as pharmaceuticals, cosmetics, or quasi-drugs (medicinal cosmetics), which are intermediate positions thereof, and can advocate various effects and efficacies.

In the present specification, singular terms may be replaced with plural terms, and vice versa, to interpret the description unless it is otherwise stated or obvious for a person skilled in the art that such replacement is impossible.

In the present specification, “consist(ing) of” means a so-called closed provision that does not include other elements, and “comprise”, “contain”, “include” and the like means a so-called open provision that can included other elements.

### EXAMPLES

### Example 1

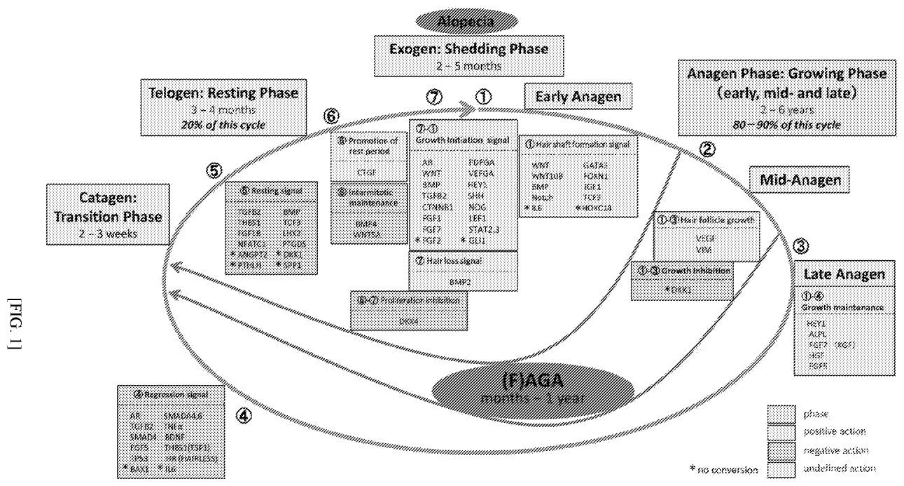

We targeted 178 hair cycle-related genes involved in the hair cycle including anagen, catagen, telogen and exogen, and according to the following protocols, we investigated whether mutations contained in genes of mesenchymal stem cells (MSCs) differentiated from iPS cells (iPSC) could be converted to wild type, that is, whether the state of the genome sequence could be improved, by introducing miR-520d-5p into the iPSCs in vitro. The 178 hair cycle-related genes were confirmed by NGS analysis (see FIG. 1).

### (1) Materials and Methods

### Cell Preparation

We used three cell lines and lentiviral vectors for DNA extraction. hiPSCs (HPS0002: 253G1) and MSCs (hMSC) were provided by the RIKEN BioResource Center Cell Bank (Nakagawa, M., et al. Generation of induced pluripotent stem cells without Myc from mouse 312 and human fibroblasts. Nat. Biotechnol. 26, 101-106 (2008).) and the Cell Resource Center for Biomedical Research, Institute of Development, Takara Bio (Kusatsu, Shiga, Japan), respectively. hiPSC (253G1) and hMSC were cultured in ReproStem medium (ReproCell, Tokyo, Japan) with 10 ng/ml of bFGF-2 and Mesenchymal Stem Cell Growth Medium (Takara Bio, Kusatsu, Shiga, Japan), respectively. Additionally, the human mesangial cell line 293FT (Invitrogen Japan K.K., Tokyo, Japan) was used to produce 520d-5p expressing-lentiviral particles for transfecting hMSCs. The 293FT cells were cultured in DMEM supplemented with 10% FBS, 0.1 mM MEM nonessential amino acid, 2 mM L-glutamine, and 1% penicillin/streptomycin. Also, we induced hMSCs from iPSCs using a STEMdiff Mesenchymal Progenitor kit (STEMCELL Technologies, Seattle, WA, USA). The hMSCs transfected by miR-520d-5p were defined as 520d/hMSCs.

### Lentiviral Vector Construct

We examined the effect of miR-520d-5p overexpression by transfecting 293FT cells (5×10⁶ cells/10-cm culture dish) with 20 μg of pMIRNA1-miR-520d-5p/GFP (System Biosciences, Mountain View, CA, USA) or the mock vector pCDH. We investigated hMSCs with miR-520d-5p expression by centrifuging the cells at 170,000×g at 4° C. for 120 min to harvest the viral particles produced in the 293FT cell culture media. The viral pellets were collected, and viral copy numbers were measured with a Lenti-X™ qRT-PCR Titration kit (Clontech, Mountain View, CA, USA). Meanwhile, one million lentiviral copies were used per 10-cm culture dish for 293FT cell infection. We transfected 50 nM synthesized oligonucleotides into 293FT cells with the FuGENE HD Transfection Reagent (Roche Diagnostics, Basel, Switzerland). pCDH/lenti/GFP treated cells were used as controls. Lastly, 520d-5p-transfected hMSCs were generated by the lentiviral transfection of 520d-5p to hMSCs after the differentiation of iPSCs to hMSCs was induced using a hiPSC-derived MSC differentiation system and reagents (Veritas Corporation, Tokyo, Japan).

### Next-Generation Sequencing (NGS) Analysis

iPSC (253G1), 520d-transfected and 253G1-derived progenitor hMSC (520d/hMSC), and hMSC were used in NGS analysis. DNA extraction was performed using a Qiagen DNeasy kit (QIAGEN, Tokyo, Japan) according to manufactures instructions. Exome sequencing was performed with a next-generation sequencer (Illumina, Inc., San Diego, USA). Three replicates per group were analyzed (n=3). Genomic DNA was processed using SureSelectXT Human All Exon v5+UTRs (Agilent Technologies, Inc.) and sequenced on the Illumina HiSeq 2500 platform with 101-bp paired-end reads (HSS, Sapporo, Japan). The paired-end reads in FastQ format were mapped to the reference genome HG19 using BWA-0.7.10. The mapping files in the SAM (Sequence Alignment/Map) format were converted to the BAM (binary version of SAM) format and sorted by SAMtools-1.2. Local realignment around the known indels was performed by GATK-Lite-2.3.0 on the sorted BAM files. Picardtools-1.133 (Nielsen, R., Paul, J. S., Albrechtsen, A., & Song, Y. S. Genotype and SNP calling from next-generation sequencing data. Nat. Rev. Genet. 12, 443-451 (2011). Wysoker, A., Tibbetts, K., & Fennell, T. picardTools 1.5, vol. 3 (2011). available: http://sourceforge.net/projects/picard/files/picard-tools/.) was used to remove PCR duplicates. Finally, base quality score recalibration was performed using GATK. RNA sequencing was performed with a next-generation sequencer (Illumina). Total RNA was processed using the TruSeq Stranded mRNA Library Prep Kit (Illumina) and sequenced on the Illumina HiSeq 2500 platform with 101 bn paired-end reads. Lastly, the paired-end reads in FastQ format were mapped to reference genome HG19 using TopHat.

### Mutation Analysis

Samtools mpileup [options: -d 10000-L 10000-B -t DP, DV, SP, DP4, DPR] was piped with beftools call [options: -A -v -m -f GQ] to produce variant call format (VCF) files. The VCF files were further filtered to select the variants with at least two supporting reads, a minimum allele frequency of 0.8, and a maximum read depth of 35 at the called sites. For high-throughput, the deep sequencing of selected cDNA was subjected to Sanger sequencing to screen for nonsynonymous mutations (Falk, M. J., et al. Mitochondrial disease genetic diagnostics: optimized whole-exome analysis for all MitoCarta nuclear genes and the mitochondrial genome. Discov. Med. 14, 389-399 (2012). Kawazu, M., et al. Transforming mutations of RAC guanosine triphosphatases in human cancers. Proc. Natl. Acad. Sci. USA. 110, 3029-3034 (2013). Takahashi, Y., Mori, J., & Kami, M. BRAF mutations in hairy-cell leukemia. N. Engl. J. Med. 365, 960-961 (2011).).

### (2) Results and Discussion

Table 2 shows the results of the nucleotide sequence conversion effects using miR-520d-5p targeting hair cycle-related genes.

For 142 of the 178 hair cycle-related genes, genomic conversion was observed to repair (at least a portion of) the mutated bases to the wild type by treating the cells with miR-520d-5p. There were 26 genes in which all of the mutated bases were repaired to the wild type, that is, the conversion rate was 100%. As an example, regarding the AR gene, 71.4% of the bases mutated in the iPSCs (5 bases out of 7 bases) returned to the wild-type bases in 520 d/h MSCs, whereas repeated sequences (22 CAG repeats and 17 GGC repeats) contained in the AR gene of the 520 d /h MSCs had the same base sequence as the base sequence in the iPSCs. Contiguous 2-base, 3-base or 4-base conversions were confirmed with 33, 8 and 1 genes, respectively (2bc, 3bc or 4bc in Table 2).

On the other hand, for 36 of the 178 hair cycle-related genes (gray background color in Table 2), no genomic conversion to repair the mutated bases to the wild type was observed by treating the cells with miR-520d-5p. However, 6 of the 36 genes did not originally have a base mutated from the wild type in iPSC (253G1).

Cells treated with pCDH/lenti/GFP used as a control did not induce genomic conversion. The transfection with miR-520d-5p showed no apparently worsening changes at the nucleotide level. The average conversion rate of 178 hair cycle-related genes was 54.4%.

TABLE 2
Summary of hair cycle-related genes with/without
conversion effects of nucleotide by 520d-5p

| | Conversion rate | Number of | |
|---|---|---|---|
| Gene | to wild type (%) | mutations | Remarks |
| ACT1 | 80.9 | 21 | |
| ALPP | 21.4 | 14 | 2bc |
| ALX4 | 13.6 | 22 | |
| ANGPT | 0 | 14 | |
| APOE | 0 | 1 | |
| AQP3 | 77.8 | 18 | 3bc |
| AR | 71.4 | 7 | |
| ASCL1 | 83.3 | 12 | |
| ASCL4 | 0 | 0 | |
| BAX | 0 | 4 | |
| BCL2 | 93.8 | 16 | |
| BDNF | 78.3 | 23 | |
| BMI1 | 98 | 49 | |
| BMP2 | 83.9 | 31 | |
| BMP4 | 50 | 14 | |
| BMP6 | 80 | 5 | |
| CCHCR1 | 15 | 20 | |
| CD200 | 0 | 3 | |
| CD271 (NGFR) | 92.3 | 13 | 2bc |
| CD34 | 0 | 0 | |
| CDH1 | 0 | 26 | |
| CDH3 | 0 | 15 | |
| CDKN1C | 100 | 10 | |
| CDKN2A | 33.3 | 3 | |
| Chr. 20 1122 PAX1 | 100 | 9 | |
| CLCA2 | 0 | 9 | |
| CPE | 60.9 | 23 | |
| CRHR2 | 82.8 | 29 | |
| CSTA | 0 | 3 | |
| CTGF | 63.3 | 8 | |
| CTNNB1 | 66.7 | 12 | 2bc |
| CYP1B1 | 64.7 | 17 | |
| DDR2 | 58.3 | 12 | |
| DIO2 | 0 | 7 | |
| DKK1 | 0 | 5 | |
| DKK2 | 100 | 10 | |
| DKK4 | 100 | 2 | |
| DLL1 | 74.1 | 27 | |
| EDA2R | 0 | 9 | |
| EDAR | 0 | 16 | |
| EDN3 | 100 | 5 | |
| EFNA | 97.5 | 40 | 2bc/3bc |
| EGF | 50 | 4 | |
| EGR1 | 100 | 18 | |
| EGR3 | 93 | 86 | 2bc |
| ENG (CD105) | 0 | 0 | |
| FBN2 | 31.8 | 22 | |
| FGF2 | 0 | 1 | |
| FGF5 | 100 | 21 | 2bc |
| FGF7 (KGF) | 66.7 | 3 | |
| FGF18 | 92.7 | 14 | |
| FN1 | 34 | 50 | 2bc |
| FOXE1 | 54.5 | 22 | |
| FOXN1 | 33.3 | 18 | 3bc |
| FOXO3 | 100 | 7 | |
| FZD1 | 100 | 50 | |
| FZD4 | 85.7 | 15 | |
| FZD8 | 94.1 | 17 | |
| GADD45G | 33.3 | 3 | |
| GATA3 | 81.3 | 16 | |
| GLI1 | 0 | 10 | |
| GLI2 | 50 | 26 | |
| GLI3 | 100 | 11 | |
| GPRC5B | 0 | 2 | |
| GRID1 | 50 | 8 | |
| GUCY1A3 | 46.7 | 15 | |
| HES1 | 100 | 18 | |
| HES5 | 85.7 | 7 | |
| HEY1 | 25 | 4 | |
| HEY2 | 90.9 | 11 | |
| HEYL | 4 | 5 | |
| HGF | 40.7 | 27 | 2bc |
| HR (HAIRLESS) | 20 | 20 | |
| HSD3B1 | 72.7 | 11 | 2bc |
| IFITM3 | 0 | 8 | |
| IGF1 | 100 | 1 | |
| IL6 | 0 | 0 | |
| IRF4 | 27.3 | 22 | 2bc |
| ITGB1 | 47.6 | 21 | |
| JAG1 | 75.6 | 41 | |
| JAG2 | 89.3 | 28 | 2bc |
| KAT2A | 95.5 | 22 | 2bc |
| KAT2B | 67.9 | 49 | |
| KCNJ8 (KIR6) | 100 | 4 | |
| KDR | 69.7 | 33 | 2bc |
| KRT5 | 100 | 25 | |
| KRT14 | 4 | 10 | |
| KRT15 | 40 | 6 | 2bc |
| KRT19 | 63.1 | 19 | 2bc |
| LAMA5 | 0 | 72 | |
| LAMC3 | 66.7 | 9 | |
| LRIG1 | 77.8 | 36 | 4bc |
| LATS2 | 38.9 | 18 | |
| LEF1 | 54.5 | 11 | |
| LFNG | 72.7 | 11 | 2bc/3bc |
| LGR5 | 50 | 12 | |
| LHX2 | 100 | 12 | |
| LMO1 | 69.6 | 23 | 2bc |
| LPL | 50 | 4 | |
| LRP4 | 62.5 | 24 | 2bc/3bc |
| LRP5 | 66.7 | 33 | |
| MAPK1 | 87.5 | 8 | 3bc |
| MKI67 (Ki67) | 0 | 29 | |
| NANOGNB (HOXC14) | 0 | 0 | |
| NCAM1 | 84.1 | 63 | 2bc/3bc |
| NCOA3 | 66.7 | 9 | |
| NCOA4 | 0 | 2 | |
| NDP | 100 | 2 | |
| NELL2 | 100 | 1 | |
| NFATC1 | 18.2 | 11 | |
| NFKB | 84.6 | 13 | |
| NOG | 100 | 53 | 2bc |
| NOS3 | 100 | 6 | |
| NOTCH2 | 45.9 | 73 | 2bc |
| NOTCH3 | 100 | 16 | 2bc |
| NOTCH4 | 25 | 28 | 2bc |
| NTRK | 82.4 | 17 | |
| PDGFA | 78.4 | 51 | |
| PTCH1 | 71.5 | 39 | |
| RBPJ | 96.2 | 27 | 2bc |
| PRSS53 | 0 | 1 | |
| PTGDS | 0 | 2 | |
| PTHLH | 0 | 2 | |
| RGS2 | 66.7 | 3 | |
| RHOB | 100 | 22 | 2bc |
| RNF144A | 0 | 5 | |
| RUNX1 | 80.8 | 26 | 2bc |
| SDKN2A | 33.3 | 3 | |
| SERPINE2 | 16.7 | 6 | |
| SHH | 94 | 50 | 2bc |
| SHISA2 | 0 | 0 | |
| SIRT7 | 60 | 5 | |
| SLC25A37 (MSCP) | 66.7 | 6 | |
| SMAD4 | 50 | 8 | |
| SMAD6 | 85.3 | 34 | |
| SMO | 27.2 | 14 | |
| SMOC2 | 100 | 2 | |
| SMTN | 33.3 | 15 | |
| SOSTDC1 | 90.9 | 11 | |
| SOX2 | 50 | 2 | |
| SOX9 | 94.7 | 57 | 2bc/3bc |
| SPECC2 | 0 | 36 | |
| SPON1 | 86.7 | 15 | |
| SPP1 | 0 | 6 | |
| SPRY1 | 0 | 1 | |
| SPRY2 | 91.7 | 24 | |
| SP5 | 77.8 | 9 | 2bc |
| SRD5A1 | 0 | 1 | |
| SRD5A2 | 20 | 15 | |
| STAR | 0 | 2 | |
| STAT2 | 0 | 0 | |
| STAT3 | 85.7 | 14 | |
| STS | 33.3 | 3 | |
| SUR2 (ABCC9) | 22.2 | 9 | |
| TCF7 | 57.1 | 7 | |
| TFAP2A | 86.7 | 45 | |
| TFAP2C | 66.7 | 3 | |
| TGFB1 | 100 | 15 | |
| TGFB2 | 100 | 14 | |
| TGFB1I1 | 92.9 | 14 | |
| THBS1 (TSP1) | 50 | 32 | |
| TIMP2 | 69.2 | 13 | |
| TRPS1 | 93.2 | 74 | 2bc |
| TWIST1 | 100 | 10 | |
| TWIST2 | 100 | 16 | 2bc |
| TYRO3 | 14.3 | 7 | |
| VAV3 | 95.2 | 21 | |
| VCAN | 75 | 40 | |
| VDR | 27.3 | 11 | |
| VEGFA | 88 | 50 | 2bc |
| VIM | 100 | 12 | |
| VSIG8 | 10 | 10 | |
| WIF1 | 0 | 2 | |
| WNT4 | 25 | 4 | |
| WNT5A | 81.8 | 22 | |
| WNT7B | 94.4 | 19 | 2bc |
| WNT9B | 94.7 | 18 | |
| WNT10B | 33.3 | 3 | |

2bc: Gene with 2 bases conversion
3bc: Gene with 3 bases conversion
4bc: Gene with 4 bases conversion

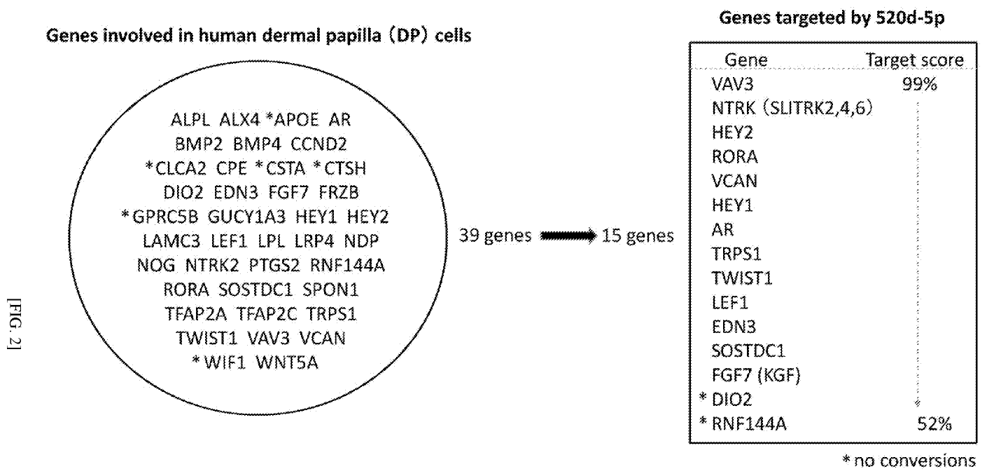

Next, for 39 genes involved in the growth or function of dermal papilla cells (DP cells) (see Non Patent Literature 1, FIG. 2), we analyzed the effect of converting the base sequence using miR-520d-5p in more detail.

miR-520d-5p improved the sequence status of 35 of the 39 genes in DP cells. Based on a target score greater than 50%, which corresponds to a 7-base match (complementary) to a mRNA sequence, miR-520d-5p potentially targets mRNA of 15 genes and suppress or regulate the expression of these genes, and miR-520d-5p improved the sequence status of 13 of the 15 genes. Interestingly, miR-520d-5p strongly targets the mRNA of the AR gene: AR and ARA55 (TGFB1 induced transcription 1) are overexpressed in the hair parts and dermal papillae of patients with male androgenetic alopecia (AGA) and female androgenetic alopecia (FAGA), and the function of the overexpressed AR can be suppressed. Simultaneously, miR-520d-5p converts mutations accumulated in the second exon of AR gene to the wild type. In DR cells, AR gene is an important element involved in (F)AGA and is regulated via various signaling pathways. AR gene is promoted by EGF, TGFβ1, NCOA4 and RUNX1 signaling and inhibited by the Notch pathway (see Non Patent Literatures 2 and 3), and it was found that miR-520d-5p can ameliorate mutations of those genes.

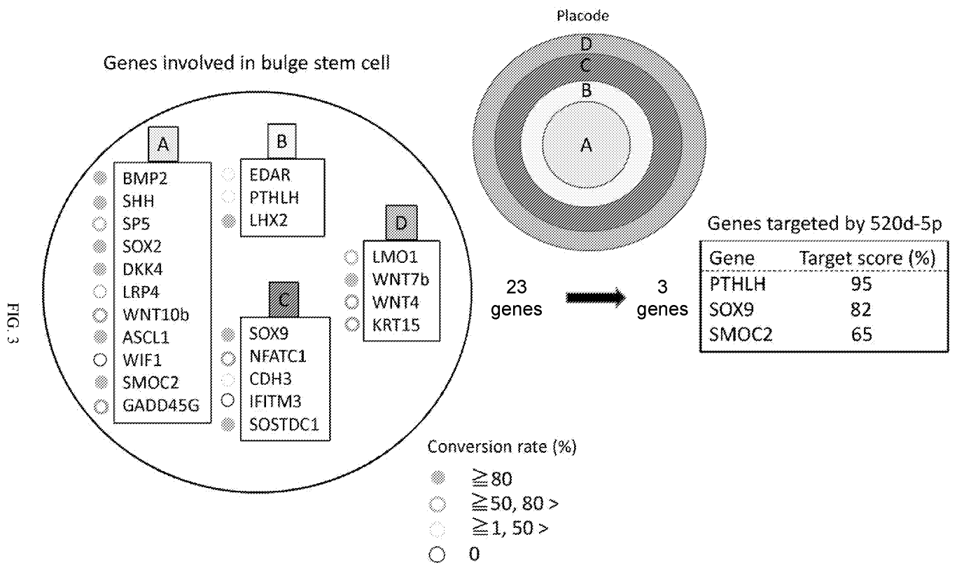

We also analyzed the effect of converting the base sequences using miR-520d-5p in more detail for 23 genes involved in the growth or function of bulge stem cells (see FIG. 3). It was revealed that the status of the sequence of 21 of those genes was improved. Based on a target score greater than 50%, miR-520d-5p potentially targets mRNAs of 3 bulge stem cell-related genes, SOX9, THLH and SMOC2, and suppress or regulate the expression of these genes. SOX9, NFATC1, LHX2, and KRT15 are known as adult stem cell markers for bulge stem cells (see Non Patent Literatures 4 to 8), and it was revealed that miR-520d-5p can ameliorate mutations in these genes.

Based on the results of Example 1 above, the action and effects of the introduction of the miR-520d-5p polynucleotide or the polynucleotide encoding the same (or the compound for upregulating miR-520d-5p) into the hair cycle-related cells such as hair papilla cells, skin-derived precursor cells, bulge stem cells and the like can repair mutations in the hair cycle-related genes or other genes expressed in those cells, and can further suppress the expression of mRNA of some hair cycle-related genes, is supported.

### Example 2

We investigated whether the expression of miR-520d-5p is upregulated by introducing a compound for upregulating miR-520d-5p into normal human skin fibroblasts (NHDFs) in vitro according to the following procedure.

Solutions were prepared using the test compounds and the solvents shown in Table 3. The solutions were added to a medium in an amount such that the final concentration in the medium was the concentrations shown in Table 3, and NHDF cells were cultured for 7 to 14 days. The adult NHDF cells were purchased from TAKARA BIO Inc. (Tokyo, Japan).

TABLE 3

| | | Final concentration | | |
|---|---|---|---|---|
| | Test compounds | in the medium | | Solvent |
| C | (Control) | No solute | | Water |
| 1 | Vancomycin | 50 | mg/mL | Water |
| 2 | Metoprolol | 41.8 | ng/mL | Ethanol |
| 3 | Benzethonium | 36 | ng/mL | Water |
| 4 | Chlorpropamide | 50 | mg/mL | 100% DMSO |
| 5 | Betaxolol | 1.0 | mg/mL | Methanol |
| 6 | Colistin | 100 | mg/mL | Water |
| 7 | Oxprenolol | 30.18 | ng/μL | DMSO |
| 8 | Ethacrynic acid | 20 | mg/mL | DMSO |
| 9 | Bisoprolol fumarate | 100 | ng/mL | Water/Methanol |
| 10 | Candesartan | 1.18 | g/mL | DMSO |
| 11 | Alexidine | 1 | mM | Water |
| 12 | Bumetanide | 0.4 | μg/mL | 95% Ethanol |
| 13 | Glycopyrrolate | 800 | pg/mL | Water |

The expression level of miR-520d-5p in the NHDFs after the culturing was measured by adding a linker to the expressed miRNA (miR-520d-5p) and detecting the miRNA by RT-PCR method with a specific primer set. The expression level of miR-520d-5p for the test compounds 1 to 13 were used was converted into a relative value when the expression level of miR-520d-5p of the control was 1.

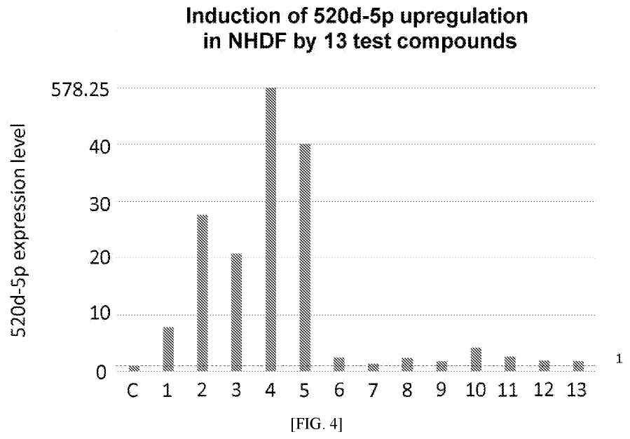

The results are shown in FIG. 4. It can be seen that all of the 13 test compounds used in this example have the effect of upregulating miR-520d-5p in NDHF. By combining the results of Example 2 with the results of Example 1, even when the compound for upregulating miR-520d-5p is introduced into hair cycle-related cells, it can be presumed that the effect of restoring the wildtype or partially restoring the wildtype is exerted, that is, the expression of −520d-5p can be enhanced, and as a result, mutations in the hair cycle-related genes and other genes expressed by the hair cycle-related cells can be repaired. In addition, it can be also presumed that the maximum effective blood concentration or the concentration in an external preparation when the compound for upregulating miR-520d-5p (13 test compounds and other compounds) is used in such applications as reverting mutations in hair cycle-related genes to the wild type, or for treating hair cycle-related diseases or symptoms, can be lower than when used as an active ingredient for other indications.

1-11. (canceled)

12. A method for producing a hair cycle-related cell, comprising:
1) a step of culturing a cell using a miR-520d-5p polynucleotide or a polynucleotide encoding miR-520d-5p polynucleotide, wherein the polynucleotide comprises:
(a1) a nucleotide sequence set forth in SEQ ID NO:1 (Single-stranded miRNA sequence) 5′-CUACAAAGGGAAGCCCUUUC-3′; or
(a2) a nucleotide sequence having 1 to 3 nucleotide deletions, substitutions, insertions or additions in the nucleotide sequence set forth in SEQ ID NO:1, or
a compound for upregulating miR-520d-5p polynucleotide, wherein the compound for upregulating miR-520d-5p is selected from the group consisting of the following compounds:
Vancomycin, Metoprolol, Benzethonium, Chlorpropamide, Betaxolol, Colistin, Oxprenolol, Ethacrynic acid, Bisoprolol, Alexidine, Bumetanide and Glycopyrrolate, and
2) a step of identifying whether a mutation in a hair cycle-related gene is reverted to wild type in the cell.

13-15. (canceled)

16. A method of reverting a mutation in a hair cycle-related gene to the wild type in a cell of a patient in need, comprising administering to the patient in need
a) miR-520d-5p polynucleotide;
b) a polynucleotide encoding miR-520d-5p polynucleotide; or
c) a compound for upregulating miR-520d-5p,
wherein the miR-520d-5p polynucleotide comprises:
(a1) a nucleotide sequence set forth in SEQ ID NO:1; or
(a2) a nucleotide sequence having 1 to 3 nucleotide deletions, substitutions, insertions or additions in the nucleotide sequence set forth in SEQ ID NO: 1,
wherein the compound for upregulating miR-520d-5p is selected from the group consisting of the following compounds:
Vancomycin, Metoprolol, Benzethonium, Chlorpropamide, Betaxolol, Colistin, Oxprenolol, Ethacrynic acid, Bisoprolol, Alexidine, Bumetanide and Glycopyrrolate.

17. The method according to claim 16, wherein the cell of the patient in need is a dermal papilla cell, a skin-derived precursor cell or a bulge stem cell, or a stem cell that can be differentiated into said cells.

18. The method according to claim 16, wherein the hair life cycle-related gene is at least one gene selected from the group consisting of the following 142 genes:
ACT1, ALPL, ALX4, AQP3, AR, ASCL1,
BCL2, BDNF, BMI1, BMP2, BMP4, BMP6,
CCHCR1, CD271, CDKN1C, CDKN2A, Chr.20 1122 PAX1, CPE, CRHR2, CTGF, CTNNB1, CYP1B1,
DDR2, DKK2, DKK4, DLL1,
EDN3, EFNA, EGF, EGR1, EGR3,
FBN2, FGF5, FGF7 (KGF), FGF18, FN1, FOXE1, FOXN1, FOXO3, FZD1, FZD4, FZD8,
GADD45G, GATA3, GLI2, GLI3, GRID1, GUCY1A3,
HES1, HES5, HEY1, HEY2, HEYL, HGF, HR (HAIRLESS), HSD3B1,
IGF1, IRF4, ITGB1,
JAG1, JAG2,
KAT2A, KAT2B, KCNJ8 (KIR6), KDR, KRT5, KRT14, KRT15, KRT19,
LAMC3, LRIG1, LATS2, LEF1, LFNG, LGR5, LHX2, LMO1, LPL, LRP4, LRP5,
MAPK1,
NCAM1, NCOA3, NDP, NELL2, NFATC1, NFKB, NOG, NOS3, NOTCH2, NOTCH3, NOTCH4, NTRK,
PDGFA, PTCH1,
RBPJ, RGS2, RHOB, RUNX1,
SDKN2A, SERPINE2, SHH, SIRT7, SLC25A37 (MSCP), SMAD4, SMAD6, SMO, SMOC2, SMTN, SOSTDC1, SOX2, SOX9, SPON1, SPRY2, SP5, SRD5A2, STAT3, STS, SUR2 (ABCC9),
TCF7, TFAP2A, TFAP2C, TGFB1, TGFB2, TGFB111, THBS1 (TSP1), TIMP2, TRPS1, TWIST1, TWIST2, TYRO3,
VAV3, VCAN, VDR, VEGFA, VIM, VSIG8, WNT4, WNT5A, WNT7B, WNT9B and WNT10B.

19. The method according to claim 16, wherein administration of the miR-520d-5p polynucleotide or the polynucleotide encoding the miR-520d-5p polynucleotide or the compound for upregulating miR-520d-5p suppresses or regulates the expression of at least one gene selected from the group consisting of the following 18 genes:
AR,
DIO2,
EDN3,
FGF7 (KGF),
HEY1, HEY2,
LEF1,
NTRK (SLITRK2, 4, 6),
PTHLH,
RNF144A, RORA,
SMOC2, SOSTDC1, SOX9,
TRPS1, TWIST1,
VAV3 and VCAN.

20. The method according to claim 16,
wherein the cell of the patient in need is a dermal papilla cell or a stem cell that can be differentiated into said cell, and
wherein the hair cycle-related gene is at least one gene selected from the group consisting of the following 26 genes:
ALX4, AR,
BMP2, BMP4,
CPE,
EDN3,
FGF7 (KGF),
GUCY1A3,
HEY1, HEY2,
LAMC3, LEF1, LPL, LRP4,
NDP, NOG, NTRK,
SOSTDC1, SPON1,
TFAP2A, TFAP2C, TRPS1, TWIST1,
VAV3, VCAN and WNT5A.

21. The method according to claim 16, wherein administration of the miR-520d-5p polynucleotide or the polynucleotide encoding the miR-520d-5p polynucleotide or the compound for upregulating miR-520d-5p suppresses or regulates the expression of at least one gene selected from the group consisting of VAV3, NTRK (SLITRK2, 4, 6), HEY2, RORA, VCAN, HEY1, AR, TRPS1, TWIST1, LEF1, EDN3, SOSTDC1, FGF7 (KGF), DIO2 and RNF144A.

22. The method according to claim 16, wherein the cell of the patient in need is a bulge stem cell or a stem cell that can be differentiated into said cell, and
wherein the hair cycle-related gene is at least one gene selected from the group consisting of the following 18 genes:
ASCL1,
BMP2,
DKK4,
GADD45G,
KRT15,
LHX2, LMO1, LRP5,
NFATC1,
SHH, SMOC2, SOSTDC1, SOX2, SOX9, SP5, SRD5A2, STAT3, STS, SUR2 (ABCC9),
TCF7, TFAP2A, TFAP2C, TGFB1, TGFB2, TGFB111, THBS1 (TSP1), TIMP2, TRPS1, TWIST1, TWIST2, TYRO3,
VAV3, VCAN, VDR, VEGFA, VIM, VSIG8, WNT4, WNT5A, WNT7B, WNT9B and WNT10B.

23. The method according to claim 16, wherein administration of the miR-520d-5p polynucleotide or the polynucleotide encoding the miR-520d-5p polynucleotide or the compound for upregulating miR-520d-5p suppresses or regulates the expression of at least one gene selected from the group consisting of PTHLH, SOX9 and SMOC2.

24. The method according to claim 16,
wherein the cell of the patient in need is a dermal papilla cell or a stem cell that can be differentiated into said cell and a bulge stem cell or a stem cell that can be differentiated into said cell, and
wherein the hair cycle-related gene is selected from the group consisting of the following 2 genes:
BMP2 and SOSTDC1.

25. The method according to claim 16,
wherein the miR-520d-5p polynucleotide is
a double-stranded polynucleotide constituted by
a polynucleotide having
(b1) a nucleotide sequence set forth in SEQ ID NO:2; or
(b2) a nucleotide sequence having 1 to 3 nucleotide deletions, substitutions, insertions or additions in the nucleotide sequence set forth in SEQ ID NO:2, and a polynucleotide having
(c1) a nucleotide sequence set forth in SEQ ID NO:3; or
(c2) a nucleotide sequence having 1 to 3 nucleotide deletions, substitutions, insertions or additions in the nucleotide sequence set forth in SEQ ID NO:3, or
a polynucleotide having
(d1) a nucleotide sequence set forth in SEQ ID NO:4; or
(d2) a nucleotide sequence having 1 to 6 nucleotide deletions, substitutions, insertions or additions in the nucleotide sequence set forth in SEQ ID NO:4.

26. The method according to claim 16,
wherein administration of each compound for upregulating miR-520d-5p gives a maximum effective blood concentration or is applied externally at a concentration selected from the group consisting of:
Vancomycin: administration, not more than 30 μg/ml;
Metoprolol: administration, not more than 41.8 ng/ml;
Benzethonium: external application, not more than 0.2%;
Chlorpropamide: administration, not more than 30 μg/ml;
Betaxolol: administration, not more than 41.8 ng/ml;
Colistin: administration, not more than 4.4 μg/ml;
Oxprenolol: administration, not more than 30.18 μg/ml;
Ethacrynic acid: administration, not more than 100 μg/ml;
Bisoprolol: administration, not more than 100 μg/ml;
Alexidine: administration, not more than 1 mM;
Bumetanide: administration, not more than 0.4 μg/ml; and
Glycopyrrolate: administration, not more than 800 pg/ml.

27-29. (canceled)

30. The method according to claim 12, wherein the cell is a dermal papilla cell, a skin-derived precursor cell or a bulge stem cell, or a stem cell that can be differentiated into said cells.
